(19) United States
(12) Patent Application Publication
(10) Pub. No.: US 2025/0032570 A1
(43) Pub. Date: Jan. 30, 2025

(54) METHOD FOR MANUFACTURING HEALTHY FOOD WITH EXCELLENT EFFICACY IN PREVENTING HAIR LOSS, PROMOTING HAIR GROWTH, STRENGTHENING HAIR ROOTS, BLACKENING HAIR, AND IMPROVING SCALP

(71) Applicant: Gyeongja YANG, Changwon-si (KR)

(72) Inventor: Gyeongja YANG, Changwon-si (KR)

(21) Appl. No.: 18/911,256

(22) Filed: Oct. 10, 2024

Related U.S. Application Data
(63) Continuation of application No. PCT/KR2023/009354, filed on Jul. 3, 2023.

(30) Foreign Application Priority Data
Jul. 2, 2023 (KR) 10-2023-0085431

(51) Int. Cl. A61K 36/539; A23L 7/104; A23L 11/50; A23L 19/00; A23L 19/10; A61K 9/00; A61K 36/074; A61K 36/14; A61K 36/185; A61K 36/39; A61K 36/535; A61K 36/64; A61K 36/73; A61K 36/744; A61K 36/756; A61K 36/78; A61K 36/82; A61K 36/882; A61K 36/8965; A61K 47/42; A61K 47/46

(52) U.S. Cl. CPC A61K 36/539; A23L 7/104; A23L 11/50; A23L 19/01; A23L 19/10; A61K 9/0053; A61K 36/074; A61K 36/14; A61K 36/185; A61K 36/39; A61K 36/535; A61K 36/64; A61K 36/73; A61K 36/744; A61K 36/756; A61K 36/78; A61K 36/82; A61K 36/882; A61K 36/8965; A61K 47/42; A61K 47/46; A23V 2400/11; A61K 2236/19

(57) ABSTRACT

Provided is a method of manufacturing a health functional food, which includes step (S1) of preparing a red ginseng fermentation powder mixed with red ginseng, black ginseng, ginger, bellflower, and jujube, step (S2) of preparing a medicinal powder mixed with Bikal skullcap (Scutellaria baicalensis), Amur Corktree (Phellodendron amurense), gardenia (Gardenia jasminoides), Purslane (Portulaca oleracea), Chameleon plant (Houttuynia cordata), tea plant (Camellia sinensis), Beefsteak plant (Perilla frutescens var. crispa), Cuscuta chinensis Lam, Cistanche Deserticola, Asparagus cochinchinensis, rehmania root (Rehmannia glutinosa), steamed rehmania root, Thuja Orientalis Seed (Seed of Platycladus orientalis fruit), Rubus coreanus, Reishi mushroom (Ganoderma lingzhi), and Acorus gramineu, step (S3) of preparing a grain mixture mixed with rice, white rice barley (Hordeum distichon), glutinous rice, corn, Foxtail millet (Setaria italica), black sesame, perilla, Coix lacryma-jobi, brown rice, sorghum, fermented soybean, Canavalia ensiformis, chickpea (Cicer arietinum), buckwheat, green gram (Vigna radiata), and oat, step (S4) of preparing a vegetable mixture mixed with sea bamboo shoot, carrot, burdock root, lotus root, cinnamon, water parsley (Oenanthe), broccoli, kelp, and cabbage.

### TECHNICAL FIELD

The present invention relates to a method of manufacturing health functional food that contain nutrients essential for preventing hair loss, improving the scalp, and growing hair, and can serve as a nutritionally balanced dietary alternative and health functional food.

### BACKGROUND TECHNOLOGY OF THE INVENTION

In recent years, functional products for health maintenance assistance are being developed enthusiastically in preference foods due to the improvement of the economic level and the increase in interest in health.

In particular, Seonsik (or Misugaru, korean multi-grain powder drink) is a breakfast substitute liquid food for busy modern people, and it is used as a patient recovery food and food for children or test taker who can eat without burden during study, and its demand as a health functional food for modern people is increasing day by day due to the abundance of starchy dietary fiber minerals.

On the other hand, modern people, regardless of age or sex, are experiencing a lot of hair loss symptoms and are consuming a lot of diet or health functional foods to treat or improve them.

Currently, many health functional foods advertising that they are effective in preventing hair loss and promoting hair growth are being released, but the effect is very limited and insignificant, so it does not sufficiently meet the needs of consumers with hair loss symptoms.

Korean Registered Patents No. 10-1476346, No. 10-0615987, and No. 10-1040778 disclose a method of manufacturing such a Seonsik.

However, because the pre-type Seonsik manufacturing method published in these conventional documents consists of simply drying, roasting, pulverizing, powdering, and packaging the ingredients, the texture is monotonous and does not induce chewing or digesting movement, so the secretion of amylase in the mouth is not good, and digestion is not good.

In addition, although the existing Seonsik consists of only grains in terms of ingredients and insufficiently includes vegetable powder, there were not many healthy functional selection that can effectively provide ingredients for activating the human body, thus, Development research that can effectively provide Seonsik ingredients for activating the human body and increasing immunity, especially preventing hair loss and promoting hair growth, is at a minimal level.

On the other hand, many health functional foods related to hair loss prevention are released, and long-term use can be said to prevent hair loss and promote hair growth, but the effect is very limited and insignificant, so it does not sufficiently meet the needs of consumers with hair loss symptoms.

### CONTENTS OF THE INVENTION

### Purpose of Invention

This invention was devised to solve the above problem, and the purpose of this invention is to provide a health functional food manufacturing method that contains nutrients essential for preventing hair loss, improving the scalp, and growing hair, and can serve as a nutritionally balanced dietary alternative and health functional food.

### Solution to the Problem

In order to achieve the above purpose, in the method of manufacturing health functional food, Method for manufacturing healthy food according to the present invention comprise:
Step (S1) of preparing a red ginseng fermentation powder, wherein after mixing red ginseng, black ginseng, ginger, bellflower, and jujube, and then boiling them in water, oligosaccharide, marine collagen, a collagen ingredient extracted from fish, and EM fermentation solution were added to ferment for a predetermined time, dried, and then powdered;
Step (S2) of preparing a medicinal powder, wherein medicinal ingredients mixed with Bikal skullcap (Scutellaria baicalensis), Amur Corktree (Phellodendron amurense), gardenia (Gardenia jasminoides), Purslane (Portulaca oleracea), Chameleon plant (Houttuynia cordata), tea plant (Camellia sinensis), Beefsteak plant (Perilla frutescens var. crispa), Cuscuta chinensis Lam, Cistanche Deserticola, Asparagus cochinchinensis, rehmania root (Rehmannia glutinosa), steamed rehmania root, Thuja Orientalis Seed (Seed of Platycladus orientalis fruit), Rubus coreanus, Reishi mushroom (Ganoderma lingzhi), and Acorus gramineus in a predetermined weight percent is put into the EM fermentation solution, fermented at room temperature for 72 hours, and then taken out, steamed with water vapor at 85 to 100° C. for 4 hours, dried at 60 to 80° C. for 24 hours, and then powdered;
Step (S3) of preparing a grain mixture, wherein after washing rice, white rice barley (Hordeum distichon), glutinous rice, corn, Foxtail millet (Setaria italica), black sesame, perilla, Coix lacryma-jobi, brown rice, sorghum, fermented soybean, Canavalia ensiformis, chickpea (Cicer arietinum), buckwheat, green gram (Vigna radiata), and oat, a mixture made by mixing them in a predetermined weight percent is put into EM fermentation solution, fermented at room temperature for 48 hours, dried, and then powdered;
Step (S4) of preparing a vegetable mixture, wherein after washing and slicing sea bamboo shoot, carrot, burdock root, lotus root, cinnamon, water parsley (Oenanthe), broccoli, kelp, and cabbage, a mixture made by mixing them in a predetermined weight percent is put into EM fermentation solution, fermented at room temperature for 48 hours, dried, and then powdered; and
Step (S5) of mixing the red ginseng fermentation powder, the medicinal powder, the grain mixture, and the vegetable mixture in a predetermined ratio.

As a preferred embodiment of this invention, Wherein the EM fermentation solution is manufactured by mixing 40 wt % of probiotic bacteria, 30 wt % of lactobacillus, and 30 wt % of Bacillus bacteria, and then incubating at 30˜50° C. for 7 days to prepare an EM raw solution, adding a predetermined amount of pulverized Turmeric (Curcuma longa) and oligosaccharide per liter of EM raw solution to the EM raw solution, and then fermenting it for a predetermined period of time.

### Effects of the Invention

Health functional foods such as the Seonsik manufactured by the manufacturing method of the present invention include various grains and vegetables, as well as additional mixed powders containing various fermentation health functional ingredients, and can also serve as health functional foods such as promoting hair growth and increasing immunity to prevent hair loss.

### BRIEF DESCRIPTION OF THE DRAWINGS

FIG. 1 is a block diagram schematically illustrating a method of preparing a health functional food according to the present invention.

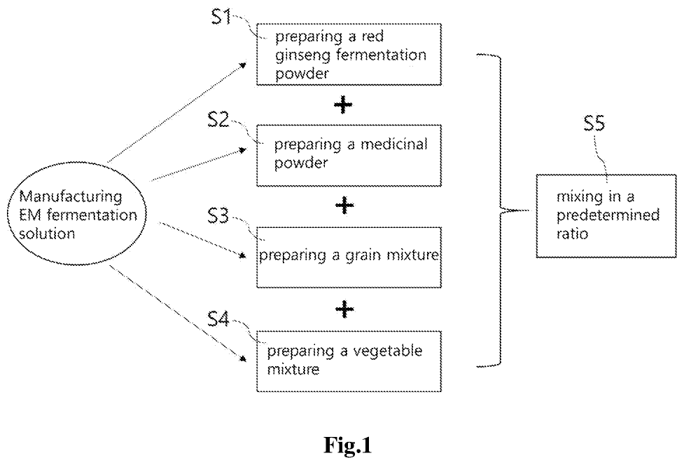

### DETAILED DESCRIPTION

The step (S1) of manufacturing a red ginseng fermentation powder of the present invention is to prepare red ginseng fermentation powder by mixing red ginseng, black ginseng, ginger, bellflower, and jujube in the same weight percent (ratio), boiling them in water, adding oligosaccharides, marine collagen, a collagen component extracted from fish, and EM fermentation solution, fermenting them for a predetermined time, drying them, and pulverizing them to produce red ginseng fermentation powder.

In an embodiment, the time to boil with water in step S1 is 8 to 10 hours, and after boiling in water and cooling sufficiently at room temperature, when the water temperature is around 35° C., 300 g of oligosaccharide, 200 g of marine colakene, and 0.01l of EM fermentation solution is added per 1l of the mixture, fermented for 48 hours, dried with a known dryer, and powdered with a known powder maker to prepare red ginseng fermentation powder.

The marine collagen is collagen extracted from the skin of fish, so it has the effects of animal collagen as it is. That is, marine collagen is effective in improving the scalp and nourishing the hair.

The step (S2) of manufacturing a medicinal powder of the present invention is to put the medicinal ingredients mixed with Bikal skullcap (Scutellaria baicalensis), Amur Corktree (Phellodendron amurense), gardenia (Gardenia jasminoides), Purslane (Portulaca oleracea), Chameleon plant (Houttuynia cordata), tea plant (Camellia sinensis), Beefsteak plant (Perilla frutescens var. crispa), Cuscuta chinensis Lam, Cistanche Deserticola, Asparagus cochinchinensis, rehmania root (Rehmannia glutinosa), steamed rehmania root, Thuja Orientalis Seed (Seed of Platycladus orientalis fruit), Rubus coreanus, Reishi mushroom (Ganoderma lingzhi), and Acorus gramineus in a predetermined weight percent (ratio) into the EM fermentation solution, ferment at room temperature for 72 hours, take out, steam it for 4 hours with water vapor at 85 to 100° C., dry it at 60 to 80° C. for 24 hours, and powder it to prepare the medicinal powder.

As an embodiment, in step S2, the Bikal skullcap, Amur Corktree, gardenia, Purslane, Chameleon plant, tea plant, Beefsteak plant, Cuscuta chinensis Lam, Cistanche Deserticola, Asparagus cochinchinensis, rehmania root, steamed rehmania root, Thuja Orientalis Seed, Rubus coreanus, Reishi mushroom, and Acorus gramineus may be mixed in the same weight ratio (percent).

In a preferred embodiment, the EM fermentation solution of the present invention may be prepared by mixing 40 wt % of probiotic bacteria, 30 wt % of lactobacillus (lactic acid bacteria), and 30 wt % of Bacillus bacteria, and then incubating (culturing) at 30 to 50° C. for 7 days to prepare an EM raw solution, and then adding 500 g of pulverized Turmeric (Curcuma longa) and 300 g of oligosaccharide to 1 l of the EM raw solution, and fermenting it for 10 days.

The step (S3) of manufacturing the grain mixture of the present invention is to prepare a grain mixture by washing rice, white rice barley (Hordeum distichon), glutinous rice, corn, Foxtail millet (Setaria italica), black sesame, perilla, Coix lacryma-jobi, brown rice, sorghum, fermented soybean, Canavalia ensiformis, chickpea (Cicer arietinum), buckwheat, green gram (Vigna radiata), and oat, fermenting at room temperature for 48 hours a mixture made by mixing them in a predetermined weight percent, drying the mixture, and pulverizing it.

As an embodiment, in step S3, rice, white rice barley, glutinous rice, corn, Foxtail millet, black sesame, perilla, Coix lacryma-jobi, brown rice, sorghum, fermented soybean, Canavalia ensiformis, chickpea, buckwheat, green gram, and oat may be mixed in the same weight ratio, and a known method may be adopted as the drying method and the powdering method.

The step (S4) of manufacturing the vegetable mixture of the present invention is to prepare a vegetable mixture by washing and chopping sea bamboo shoot, carrot, burdock root, lotus root, cinnamon, water parsley (Oenanthe), broccoli, kelp, and cabbage, and then putting a mixture made by mixing them in a predetermined weight percent into an EM fermentation solution, fermenting it at room temperature for 48 hours, drying it, and pulverizing it.

As an embodiment, in step S4, the sea bamboo shoot, carrot, burdock root, lotus root, cinnamon, water parsley, broccoli, kelp, and cabbage may be mixed in the same weight ratio.

As a desirable embodiment of the step (S5) of this invention, the red ginseng fermentation powder, the medicinal powder, the grain mixture, and the vegetable mixture may be mixed in a ratio of 1:1:6:2 to produce a Seonsik.

### EMBODIMENT OF THE INVENTION

Hereinafter, a preferred embodiment of the present invention will be described, and the efficacy of Seonsik (Drink), which is a kind of health functional food prepared by the embodiment, will be confirmed.

### Embodiment

First, 40 wt % of probiotic bacteria, 30 wt % of lactobacillus (lactic acid bacteria), and 30 wt % of Bacillus bacteria are mixed in a container and then cultured at 30 to 50° C. for 7 days to prepare an EM raw solution. Thereafter, 50 g of pulverized Turmeric (Curcuma longa) and 20 g of oligosaccharide per 1 liter of the EM raw solution are added and fermented for 10 days to prepare an EM fermentation solution.

In addition, 50 g of red ginseng, 50 g of black ginseng, 50 g of ginger, 50 g of bellflower, and 50 g of jujube that have been washed in water are mixed and boiled in 10 of water, 10 g of oligosaccharide, 20 g of marine collagen, a collagen component extracted from fish, and 0.10 of EM fermentation solution are added to ferment for 10 days, dried through a known dryer, and then powdered through a known powder machine to prepare red ginseng fermentation powder.

Next, the medicinal ingredients mixed with Bikal skullcap (Scutellaria baicalensis) 50 g, Amur Corktree (Phellodendron amurense) 50 g, gardenia (Gardenia jasminoides) 50 g, Purslane (Portulaca oleracea) 50 g, Chameleon plant (Houttuynia cordata) 50 g, tea plant (Camellia sinensis) 50 g, Beefsteak plant (Perilla frutescens var. crispa) 50 g, Cuscuta chinensis Lam 50 g, Cistanche Deserticola 50 g, Asparagus cochinchinensis 50 g, rehmania root (Rehmannia glutinosa) 50 g, steamed rehmania root 50 g, Thuja Orientalis Seed (Seed of Platycladus orientalis fruit) 50 g, Rubus coreanus 50 g, Reishi mushroom (Ganoderma lingzhi) 50 g, and Acorus gramineus 50 g were put in 2 liters of the EM fermentation solution, fermented at room temperature for 72 hours, then taken out, steamed with water vapor of 85-100° C. for 4 hours, dried under 70° C. for 24 hours, and powdered with a known powder to prepare the medicinal powder.

Next, after washing 50 g of rice, 50 g of white rice barley (Hordeum distichon), 50 g of glutinous rice, 50 g of corn, 50 g of Foxtail millet (Setaria italica), 50 g of black sesame, 50 g of perilla, 50 g of Coix lacryma-jobi, 50 g of brown rice, 50 g of sorghum, 50 g of fermented soybean, 50 g of Canavalia ensiformis, 50 g of chickpea (Cicer arietinum), 50 g of buckwheat, 50 g of green gram (Vigna radiata), and 50 g of oat, a mixture made by mixing them was put in 30 of EM fermentation solution, fermented at room temperature for 48 hours, dried, and pulverized with a known powder to prepare a grain mixture.

Then, after washing, respectively chopped mixture of 50 g sea bamboo shoot, 50 g carrot, 50 g burdock root, 50 g lotus root, 50 g cinnamon, 50 g water parsley (Oenanthe), 50 g broccoli, 50 g kelp, and 50 g cabbage is put in 20 of EM fermentation solution, fermented at room temperature for 48 hours, dried, and powdered with a known powder to prepare a vegetable mixture.

The red ginseng fermentation powder, the medicinal powder, the grain mixture, and the vegetable mixture were mixed at a ratio of 1:1:6:2 to prepare Seonsik powder.

### EFFICACY EXPERIMENT

### Experimental Example 1

The degree of hair loss and hair growth progression was visually evaluated when the above embodiment Seonsik manufactured according to the present invention, comparative example A Seonsik manufactured by Company A (Aram Co., Ltd. the Blue full Seonsik), and comparative example B manufactured by Company B (Taekwang Food Co., Ltd.) were taken.

Ten subjects with hair loss progression were selected, and three packs of embodiment Seonsik and comparative Seonsik were taken daily for two months, including breakfast, lunch, and dinner, and the progress of hair loss and hair growth progress were visually evaluated two weeks after taking it.

TABLE 1

| | | comparative | comparative |
|---|---|---|---|
| | Embodiment | example A | example B |
| Sortation | Seonsik | Seonsik | Seonsik |
| 30 year old male | After 20 days, hair | No effect | No effect |
| | began to form, | | |
| | and after 2 months, | | |
| | circular hair loss | | |
| | completely healed. | | |
| 58 year old male | After 25 days, new | No effect | No effect |
| | hair is created | | |
| | all over the scalp | | |
| 54 year old male | Eyebrows and hair | No effect | No effect |
| | start to grow after | | |
| | 20 days | | |
| 46 year old female | After 30 days, hair | No effect | No effect |
| | is produced | | |
| 37 year old female | After 10 days, brown | No effect | No effect |
| | hair started to | | |
| | come up and after | | |
| | 30 days, a large | | |
| | amount of hair | | |
| | was produced. | | |
| 69 year old male | After 45 days, my | No effect | No effect |
| | hair starts to come up | | |
| 51 year old male | After 30 days, hair | No effect | No effect |
| | loss stops progressing | | |
| | and then a week later, | | |
| | hair begins to rise | | |
| | from the top of the | | |
| | head | | |
| 46 year old male | Circular hair loss | No effect | No effect |
| | healing after 30 days | | |
| 65 year old female | Hair starts to break | No effect | No effect |
| | out after 45 days. | | |
| 57 year old female | After 10 days, my | No effect | No effect |
| | hands and feet warm | | |
| | up, and after 20 days, | | |
| | my eyebrows and hair | | |
| | start to appear | | |

As can be seen in Table 1 above, it was confirmed that steady consumption of Seonsik, a health functional food manufactured by the manufacturing method according to the present invention, has a hair loss treatment and hair growth effect, unlike Seonsik sold on the market such as comparative Seonsik.

### INDUSTRIAL AVAILABILITY

According to the health functional food manufacturing process of the manufacturing method of the present invention, it is possible to effectively manufacture a dietary substitute while being a health functional food having the effect of promoting hair growth and increasing immunity to prevent hair loss.

1. Method for manufacturing healthy food comprising:
Step (S1) of preparing a red ginseng fermentation powder, wherein after mixing red ginseng, black ginseng, ginger, bellflower, and jujube, and then boiling them in water, oligosaccharide, marine collagen, a collagen ingredient extracted from fish, and EM fermentation solution were added to ferment for a predetermined time, dried, and then powdered;
Step (S2) of preparing a medicinal powder, wherein medicinal ingredients mixed with Bikal skullcap (Scutellaria baicalensis), Amur Corktree (Phellodendron amurense), gardenia (Gardenia jasminoides), Purslane (Portulaca oleracea), Chameleon plant (Houttuynia cordata), tea plant (Camellia sinensis), Beefsteak plant (Perilla frutescens var. crispa), Cuscuta chinensis Lam, Cistanche Deserticola, Asparagus cochinchinensis, rehmania root (Rehmannia glutinosa), steamed rehmania root, Thuja Orientalis Seed (Seed of Platycladus orientalis fruit), Rubus coreanus, Reishi mushroom (Ganoderma lingzhi), and Acorus gramineus in a predetermined weight percent is put into the EM fermentation solution, fermented at room temperature for 72 hours, and then taken out, steamed with water vapor at 85 to 100° C. for 4 hours, dried at 60 to 80° C. for 24 hours, and then powdered;
Step (S3) of preparing a grain mixture, wherein after washing rice, white rice barley (Hordeum distichon), glutinous rice, corn, Foxtail millet (Setaria italica), black sesame, perilla, Coix lacryma-jobi, brown rice, sorghum, fermented soybean, Canavalia ensiformis, chickpea (Cicer arietinum), buckwheat, green gram (Vigna radiata), and oat, a mixture made by mixing them in a predetermined weight percent is put into EM fermentation solution, fermented at room temperature for 48 hours, dried, and then powdered;
Step (S4) of preparing a vegetable mixture, wherein after washing and slicing sea bamboo shoot, carrot, burdock root, lotus root, cinnamon, water parsley (Oenanthe), broccoli, kelp, and cabbage, a mixture made by mixing them in a predetermined weight percent is put into EM fermentation solution, fermented at room temperature for 48 hours, dried, and then powdered; and
Step (S5) of mixing the red ginseng fermentation powder, the medicinal powder, the grain mixture, and the vegetable mixture in a predetermined ratio;
Wherein the EM fermentation solution is manufactured by mixing 40 wt % of probiotic bacteria, 30 wt % of lactobacillus, and 30 wt % of Bacillus bacteria, and then incubating at 30˜50° C. for 7 days to prepare an EM raw solution, adding a predetermined amount of pulverized Turmeric (Curcuma longa) and oligosaccharide per liter of EM raw solution to the EM raw solution, and then fermenting it for a predetermined period of time.
